(19) United States
(12) Patent Application Publication
(10) Pub. No.: US 2013/0324826 A1
(43) Pub. Date: Dec. 5, 2013

(54) BIOLOGICAL SIGNAL MEASUREMENT APPARATUS, BIOLOGICAL SIGNAL MEASUREMENT EQUIPMENT, AND BIOLOGICAL SIGNAL MEASUREMENT APPARATUS SET

(71) Applicant: Sony Corporation, Tokyo (JP)

(72) Inventors: Mitsuhiro Nakamura, Kanagawa (JP); Takuro Yamamoto, Singapore (SG); Haruhiko Soma, Tokyo (JP); Tatsumi Sakaguchi, Kanagawa (JP); Tomiji Tanaka, Saitama (JP)

(73) Assignee: Sony Corporation, Tokyo (JP)

(21) Appl. No.: 13/902,185

(22) Filed: May 24, 2013

(30) Foreign Application Priority Data
Jun. 1, 2012 (JP) 2012125903

(51) Int. Cl. A61B 5/00; A61B 5/0478

(52) U.S. Cl. CPC A61B 5/6803; A61B 5/0478
USPC 600/383; 600/300

(57) ABSTRACT

A biological signal measurement apparatus includes a supporter and a vital sensor. The supporter is made of a shape-memory material. The vital sensor is configured to acquire a biological signal of a user. The vital sensor is attached to the supporter.

### BACKGROUND

The present disclosure relates to a biological signal measurement apparatus, biological signal measurement equipment, and a biological signal measurement apparatus set, that are attached to a body of a user and used for measuring biological signals of the user.

Biological signals such as brain waves, an electrocardiogram, and blood oxygen saturation can be measured by a vital sensor that is held in contact with or held close to a body of a user. Such a vital sensor is installed into equipment to be worn by the user. Then, the equipment is worn by the user. In this manner, the vital sensor can be located at a predetermined measurement position in the body of the user.

The equipment is formed in a shape conforming to the body of the user. One stably attached to the body of the user is favorable as the equipment. For example, Japanese Patent Application Laid-open No. 2011-104338 (hereinafter, referred to as Patent Document 1) discloses “biological signal measuring equipment.” In the biological signal measuring equipment, a supporter to be mounted on the head of the user installs a plurality of brain wave measurement electrodes. In this equipment, the supporter is formed in a shape curved corresponding to the shape of the head of the user.

### SUMMARY

However, it is difficult to say that the equipment disclosed in Patent Document 1 is excellent in portability due to the curved shape. In recent years, home monitoring in which biological signals such as brain waves are measured in not only medical institutions but also at home is popular. It is desirable to improve the portability of such a measurement apparatus.

In view of the above-mentioned circumstances, there is a need for providing a biological signal measurement apparatus, biological signal measurement equipment, and a biological signal measurement apparatus set, that enable a vital sensor to be reliably located at a measurement position and are excellent in portability.

According to an embodiment of the present disclosure, there is provided a biological signal measurement apparatus including a supporter and a vital sensor.

The supporter is made of a shape-memory material.

The vital sensor is configured to acquire a biological signal of a user. The vital sensor is attached to the supporter.

With this configuration, the biological signal measurement apparatus can be deformed when the user carries the biological signal measurement apparatus. Meanwhile, when the biological signal is measured, the biological signal measurement apparatus can easily recover the shape memorized in advance. Therefore, the biological signal measurement apparatus is stably attached to the body of the user and enables the vital sensor to acquire the biological signal. The biological signal measurement apparatus can be made excellent in portability when the user carries the biological signal measurement apparatus.

The supporter may memorize a shape conforming to a shape of a body of the user.

With this configuration, when the biological signal is measured, the supporter can easily recover the shape conforming to the shape of the body of the user that is memorized in advance.

The supporter may include a headband to be mounted on a head of the user. The vital sensor may include a brain wave acquiring electrode configured to abut against the head of the user and acquire the brain wave of the user.

The brain wave acquiring electrode for acquiring the brain wave is located at a predetermined position in the head of the user. For this, it is necessary to stably support the supporter (headband) on the head of the user. However, the headband stably supported on the head of the user has a three-dimensional shape, that is, becomes bulky, corresponding to the shape of the head of the user. However, the headband according to an embodiment of the present disclosure can be deformed, and hence the biological signal measurement apparatus excellent in portability can be provided.

The headband may include a first headband section extending from a forehead of the user to an upper portion of an occipital region of the user, a second headband section being connected to the first headband section and extending from the upper portion of the occipital region of the user to a right mastoid region of the user orthogonally to the first headband section, and a third headband section being connected to the first headband section and extending from the upper portion of the occipital region of the user to a left mastoid region of the user orthogonally to the first headband section.

With this configuration, the headband holds the head of the user from three sides of the headband section, the second headband section, and the third headband section, and is stably supported on the head of the user. Here, the headband according to the embodiment of the present disclosure can be deformed as described above, and the biological signal measurement apparatus can be made excellent in portability.

The first headband section, the second headband section, and the third headband section may be rotatably connected by a single rotating shaft.

With this configuration, the headband can be folded by rotating the headband sections about the rotating shaft. It is possible to further enhance the portability.

According to another embodiment of the present disclosure, there is provided biological signal measurement equipment including a supporter.

The supporter is made of a shape-memory material. A vital sensor configured to acquire a biological signal of a user can be attached to the supporter.

According to another embodiment of the present disclosure, there is provided a biological signal measurement apparatus set including a biological signal measurement apparatus and a housing casing.

The biological signal measurement apparatus includes a supporter made of a shape-memory material, and a vital sensor configured to acquire a biological signal of a user, the vital sensor being attached to the supporter.

The housing casing is configured to house the biological signal measurement apparatus and provide the supporter with a recovery condition of the shape-memory material.

As described above, according to the embodiments of the present disclosure, it is possible to provide a biological signal measurement apparatus, biological signal measurement equipment, and a biological signal measurement apparatus set, that enable a vital sensor to be reliably located at a measurement position and are excellent in portability.

These and other objects, features and advantages of the present disclosure will become more apparent in light of the following detailed description of best mode embodiments thereof, as illustrated in the accompanying drawings.

### BRIEF DESCRIPTION OF DRAWINGS

FIG. 1 is a perspective view showing a brain wave measurement apparatus according to a first embodiment of the present disclosure;

FIG. 2 is an outer appearance view showing a state of the brain wave measurement apparatus worn by a user;

FIG. 3 is an outer appearance view showing a state of the brain wave measurement apparatus worn by the user;

FIG. 4 is a schematic view showing a headband of the brain wave measurement apparatus;

FIGS. 5A and 5B are schematic views showing a change of a shape of the headband of the brain wave measurement apparatus;

FIG. 6 is a schematic view showing a brain wave measurement apparatus set according to the first embodiment of the present disclosure;

FIG. 7 is a schematic view showing a headband of a brain wave measurement apparatus according to a second embodiment of the present disclosure; and

FIGS. 8A and 8B are schematic views showing a rotating state of the headband of the brain wave measurement apparatus.

### DETAILED DESCRIPTION OF EMBODIMENTS

### First Embodiment

A brain wave measurement apparatus according to a first embodiment of the present disclosure will be described.

[Configuration of Brain Wave Measurement Apparatus]

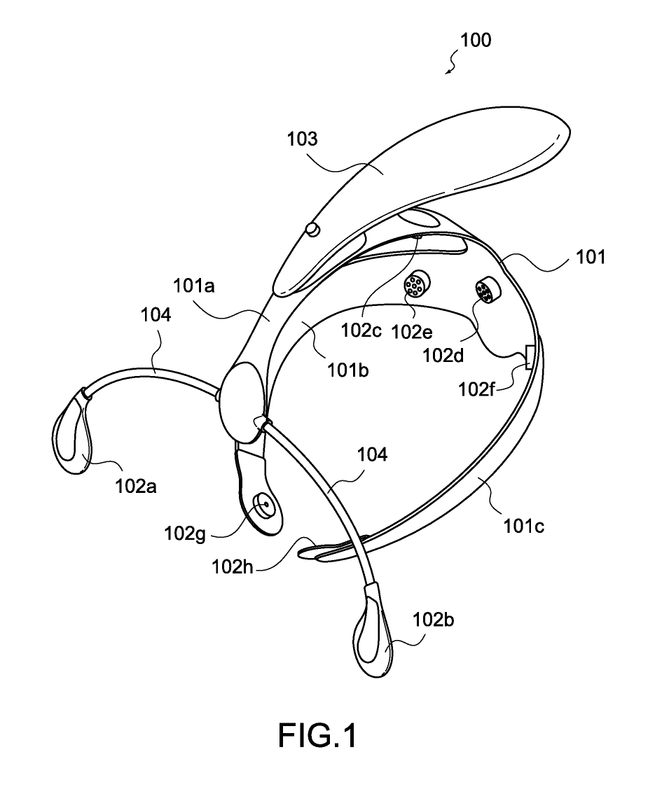

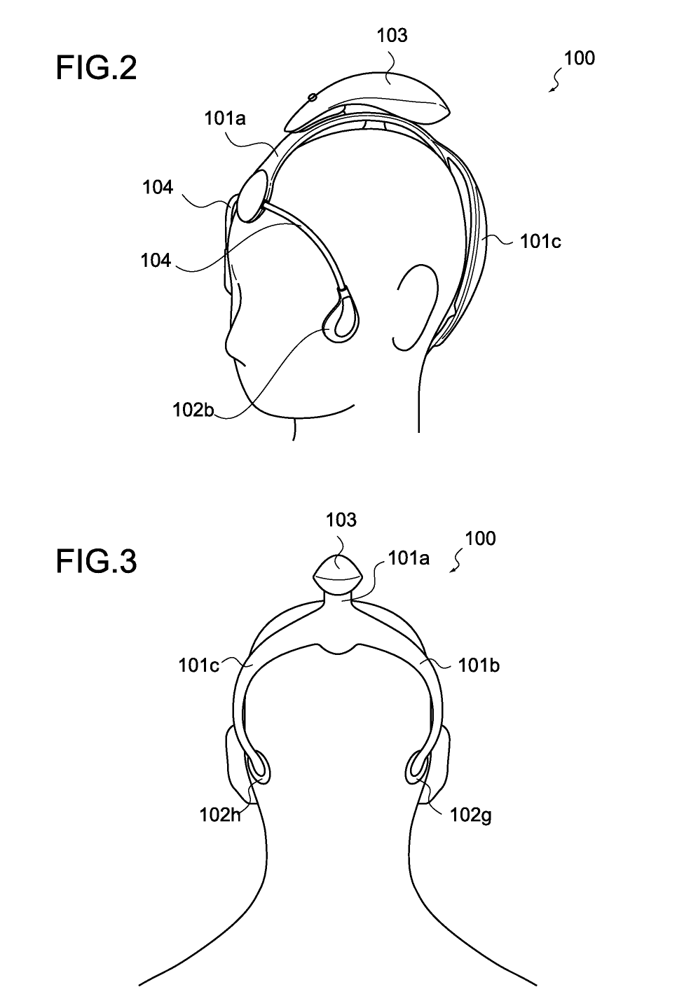

FIG. 1 is a perspective view of a brain wave measurement apparatus 100 according to the first embodiment. FIGS. 2 and 3 are outer appearance views each showing the brain wave measurement apparatus 100 worn by a user. FIG. 2 is a view as viewed from the left of the user. FIG. 3 is a view as viewed from the back of the user.

As shown in those figures, the brain wave measurement apparatus 100 includes a headband 101, electrodes 102a to 102h, and a signal processing unit 103. The electrodes 102a to 102h and the signal processing unit 103 are attached to the headband 101.

The headband 101 supports the brain wave measurement apparatus 100 on the head of the user. The headband 101 is configured to be capable of taking a shape conforming to the shape of the body of the user (referred to as use shape) and a shape for carrying (referred to as portable shape). The headband 101 will be described later in detail.

The electrodes 102a to 102h are brain wave acquiring electrodes that abut against the head of the user and acquire brain waves of the user. The electrodes 102a to 102h may be a right eye electrode 102a, a left eye electrode 102b, a Cz measurement electrode 102c, a Pz measurement electrode 102d, a P4 measurement electrode 102e, a P3 measurement electrode 102f, a right reference electrode 102g, and a left reference electrode 102h, respectively. This placement (name) complies with the International 10-20 system. However, in the brain wave measurement apparatus 100 according to this embodiment, this electrode placement do not necessarily need to be adopted and different electrode placement may be adopted depending on needs.

The right eye electrode 102a and the left eye electrode 102b are electrodes that abut against both the temples of the user to measure an electrooculogram (EGG). The right eye electrode 102a and the left eye electrode 102b may be provided to an arm 104 extending from the headband 101 to the left and right temples of the user. The right eye electrode 102a and the left eye electrode 102b only need to be capable of establishing electrical contact with the temples of the user and may be each formed of, for example, an elastic body impregnated with an electrolytic solution.

The Cz measurement electrode 102c is an electrode that is provided at a position of the headband 101 that corresponds to the parietal region, and abuts against the parietal region of the user. The Pz measurement electrode 102d is an electrode that is provided at a position of the headband 101 that corresponds to an upper portion of the occipital region, and abuts against the upper portion of the occipital region of the user.

The P4 measurement electrode 102e is an electrode that is provided at a position of the headband 101 that corresponds to the upper right head region, and abuts against the upper right head region of the user. The P3 measurement electrode 102f is an electrode that is provided at a position of the headband 101 that corresponds to the upper left head region, and abuts against the upper left head region of the user.

The Cz measurement electrode 102c, the Pz measurement electrode 102d, the P4 measurement electrode 102e, and the P3 measurement electrode 102f only need to be capable of establishing electrical contact with the scalp of the user and may be each formed of, for example, an elastic body impregnated with an electrolytic solution. The Cz measurement electrode 102c, the Pz measurement electrode 102d, the P4 measurement electrode 102e, and the P3 measurement electrode 102f detect potentials (brain waves) of their in-contact positions. Note that the number and arrangement of those measurement electrodes (electrodes 102c to 102f) are arbitrary, and those measurement electrodes may be provided depending on needs of brain wave measurement.

The right reference electrode 102g is an electrode that is provided at a position of the headband 101 that corresponds to the right mastoid region and abuts against the right mastoid region (conical protrusion located at lower rear portion of right temporal bone) of the user. The left reference electrode 102h is an electrode that is provided at a position of the headband 101 that corresponds to the left mastoid region and abuts against the left mastoid region (conical protrusion located at lower rear portion of left temporal bone) of the user. The right reference electrode 102g and the left reference electrode 102h only need to be capable of establishing electrical contact with the left and right mastoid regions of the user and may be each formed of, for example, an elastic body impregnated with an electrolytic solution. The right reference electrode 102g and the left reference electrode 102h acquire each reference potential of the electrodes 102a to 102f.

Each of the above-mentioned electrodes 102a to 102h may be electrically connected to the signal processing unit 103 via a wiring (not shown).

As described above, the signal processing unit 103 is connected to each of the electrodes 102a to 102h, processes an output from each of the electrodes 102a to 102h, generates brain waves of the user at each measurement position, and outputs the generated brain waves to an external apparatus (personal computer (PC) or the like). The signal processing unit 103 may include built-in signal processing circuit, wireless communication circuit, cell, and the like (not shown).

The generation of the brain waves in the signal processing unit 103 can be performed by known signal processing.

[Headband]

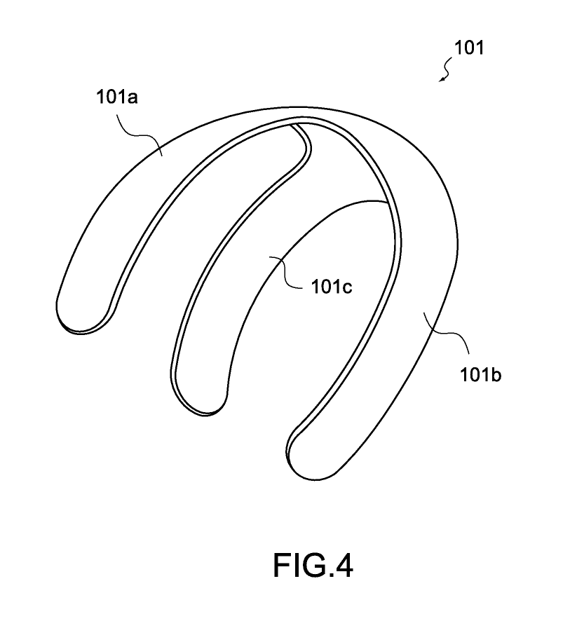

As described above, the brain wave measurement apparatus 100 is mounted on the head of the user in such a manner that the headband 101 is supported on the head of the user. Here, the head of the user has a three-dimensional shape. Therefore, by setting the headband 101 to have a three-dimensional shape corresponding to the shape of the head of the user, the headband 101 can be made stably supported on the head of the user. FIG. 4 is a schematic view showing the headband 101.

As shown in the figure, the headband 101 may include a first headband section 101a, a second headband section 101b, and a third headband section 101c.

As shown in FIGS. 2 and 3, the first headband section 101a is a plate-like member extending from the forehead of the user to the upper portion of the occipital region. The first headband section 101a is formed in a shape curved corresponding to the shape of the head.

As shown in FIGS. 2 and 3, the second headband section 101b is a plate-like member extending from the upper portion of the occipital region of the user to the right mastoid region of the user orthogonally to the first headband section 101a. The second headband section 101b is formed in a shape curved corresponding to the shape of the head.

As shown in FIGS. 2 and 3, the third headband section 101c is a plate-like member extending from the upper portion of the occipital region of the user to the left mastoid region of the user orthogonally to the first headband section 101a. The third headband section 101c is formed in a shape curved corresponding to the shape of the head.

The headband 101 is stably supported on the head of the user in such a manner that the three headband sections of the first headband section 101a, the second headband section 101b, and the third headband section 101c hold the head of the user. Further, those headband sections are connected to one another in a T-shape. Those headband sections branch to the left and the right in the upper portion of the occipital region of the user. With this shape, the headband 101 is not held in contact with a pillow or the like when the user sleeps lying on his or her back. Therefore, also while the user sleeps, it becomes possible to stably measure the brain waves.

Note that the shape of the headband 101 shown here is merely an example, and any shape may be adopted as long as the headband 101 is stably supported on the head of the user. For example, the headband 101 may include four or more headband sections connected to one another. Alternatively, the headband 101 may include two or less headband sections connected to one another. Further, the headband 101 is not limited to one constituted of the plurality of headband sections. For example, the headband 101 may be constituted of a single headband section having a curved shape extending from the forehead of the user to a lower portion of the occipital region of the user.

In the brain wave measurement apparatus 100, due to the headband 101 having a three-dimensional shape conforming to the shape of the head of the user as described above, the various electrodes 102a to 102h can be reliably located at measurement positions on the scalp of the user. However, in the brain wave measurement apparatus 100, portability becomes a problem due to the three-dimensional shape of the headband 101.

Here, the headband 101 may be made of a shape-memory material. The shape-memory material is a material deformed due to force and recovers its shape memorized in advance under a predetermined condition. Examples of the shape-memory material include a shape-memory resin and a shape-memory alloy. Hereinafter, a condition under which the shape-memory material recovers is referred to as a “recovery condition.”

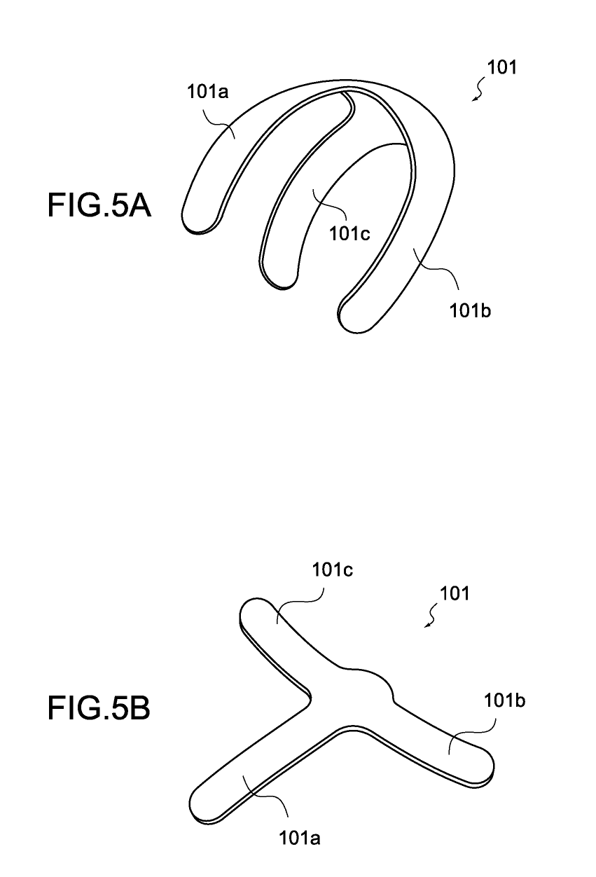

FIGS. 5A and 5B are schematic views showing a change of a shape of the headband 101. As shown in the figure, the headband 101 is capable of taking the shape (use shape) conforming to the shape of the head of the user and the flat shape (portable shape) as described above.

FIG. 5A shows the headband 101 in the use shape. FIG. 5B shows the headband 101 in the portable shape. Although FIGS. 5A and 5B show only the headband 101, the signal processing unit 103 or the various electrodes 102a to 102h may be attached or detached to/from the headband 101 upon a change of a shape of the headband 101.

The headband 101 may memorize the use shape. Using the brain wave measurement apparatus 100 with the headband 101 being in the use shape (FIG. 5A), the user can carry out brain wave measurement. After the brain wave measurement, when the user carries the headband 101, the user applies force to the headband 101 and the headband 101 can be deformed into the portable shape (FIG. 5B). Although the portable shape is not particularly limited, the portable shape can be a flat shape suitable for carrying as shown in FIG. 5B.

When the brain wave measurement is carried out again, the user provides the headband 101 with the recovery condition. With this, the headband 101 is deformed into the use shape (FIG. 5A) due to a shape recovery action by the shape-memory material of the headband 101 and can be utilized for the brain wave measurement.

The recovery condition depends on properties of the shape-memory material. The recovery condition only needs to such a condition that the shape recovery of the shape-memory material occurs, for example, heating above a predetermined temperature or cooling below a predetermined temperature.

The shape memorized by the headband 101 may be the portable shape in contrast to the above-mentioned shape. That is, the headband 101 memorizes the portable shape (FIG. 5B) and may recover the portable shape under the recovery condition. In this case, the user may apply force to the headband 101 in the portable shape and deform the headband 101 into the use shape (FIG. 5A).

In addition, both the portable shape and the use shape may be memorized by the headband 101. For example, when the headband 101 is in the use shape, the headband 101 can recover the portable shape by providing the recovery condition. When the headband 101 is in the portable shape, the headband 101 can recover the use shape by providing another recovery condition. Such a headband 101 may be realized by making the headband 101 from a plurality of kinds of shape-memory materials different in the recovery condition, for example.

As described above, the headband 101 is configured to be deformable between the use shape and the portable shape. The use shape is a three-dimensional shape conforming to the shape of the head of the user. The use shape enables the headband 101 to be stably supported on the head of the user. On the other hand, the portable shape may be a shape excellent in carrying, for example, a flat shape. Therefore, the brain wave measurement apparatus 100 can be stably mounted on the head of the user during the brain wave measurement and can be made excellent in portability.

Note that the headband 101 is not necessarily limited to one entirely made of a shape-memory material. For example, the headband 101 may be partially made of a shape-memory material at least such that the headband 101 can take the use shape and the portable shape.

[Housing Casing]

As described above, by providing the recovery condition, the headband 101 of the brain wave measurement apparatus 100 can recover from the use shape to the portable shape or from the portable shape to the use shape. Here, adding a function of realizing the recovery condition to the housing casing capable of housing the brain wave measurement apparatus 100 enables the shape of the headband 101 to be changed irrespective of a location and a time, which is highly convenient.

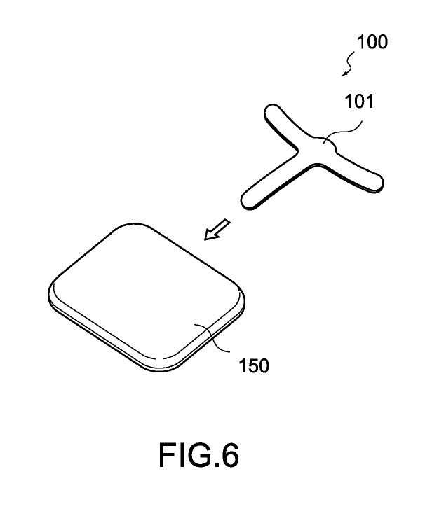

FIG. 6 is a schematic view showing the brain wave measurement apparatus 100 and a housing casing 150. As shown in the figure, in a state in which the brain wave measurement apparatus 100 is housed in the housing casing 150, the housing casing 150 provides the headband 101 with the recovery condition. In this manner, the headband 101 can recover the shape.

The housing casing 150 only needs to be capable of providing the headband 101 with the recovery condition. For example, if the recovery condition is heating above a predetermined temperature, a housing casing incorporating a heating mechanism may be adopted. Otherwise, if the recovery condition is cooling below a predetermined temperature, a housing casing incorporating a cooling mechanism may be adopted.

### Second Embodiment

A brain wave measurement apparatus according to a second embodiment of the present disclosure will be described. The brain wave measurement apparatus according to this embodiment may have the same configuration as that of the brain wave measurement apparatus according to the first embodiment except for the headband.

[Headband]

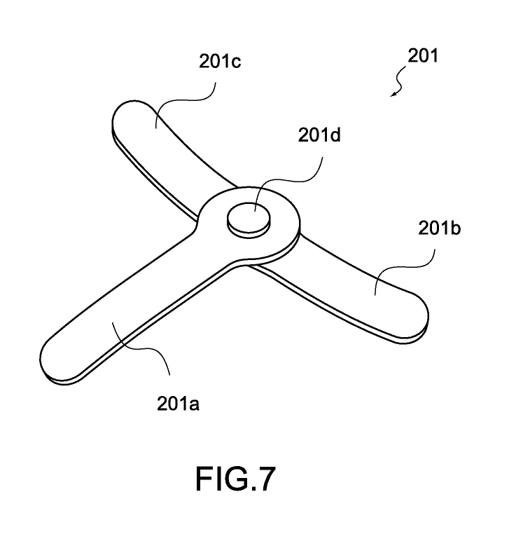

FIG. 7 is a schematic view showing a headband 201 of the brain wave measurement apparatus according to the second embodiment of the present disclosure. As in the first embodiment, the headband 201 may be made of a shape-memory material. The headband 201 is configured to be capable of taking a use shape conforming to the shape of the head of the user and a portable shape suitable for carrying. FIG. 7 shows the headband 201 in the portable shape. The use shape of the headband 201 is the same as that of the headband 101 (see FIG. 4) according to the first embodiment.

As shown in FIG. 7, the headband 201 may include a first headband section 201a, a second headband section 201b, and a third headband section 201c.

When the headband 201 takes the use shape, the first headband section 201a, the second headband section 201b, and the third headband section 201c may have a shape conforming to the shape of the head of the user as in the first embodiment. That is, the first headband section 201a may be a plate-like member extending from the forehead of the user to an upper portion of the occipital region. The second headband section 201b may be a plate-like member extending from the upper portion of the occipital region of the user to the right mastoid region of the user orthogonally to the first headband section 201a. Further, the third headband section 201c may be a plate-like member extending from the upper portion of the occipital region of the user to the left mastoid region of the user orthogonally to the first headband section 201a.

The first headband section 201a, the second headband section 201b, and the third headband section 201c may be rotatably connected by a rotating shaft 201d.

The rotating shaft 201d may be provided at a position at which the headband sections 201a to 201c are connected, that is, a position corresponding to the upper portion of the occipital region of the user. The rotating shaft 201d may extend along a thickness direction of each of the headband sections 201a to 201c.

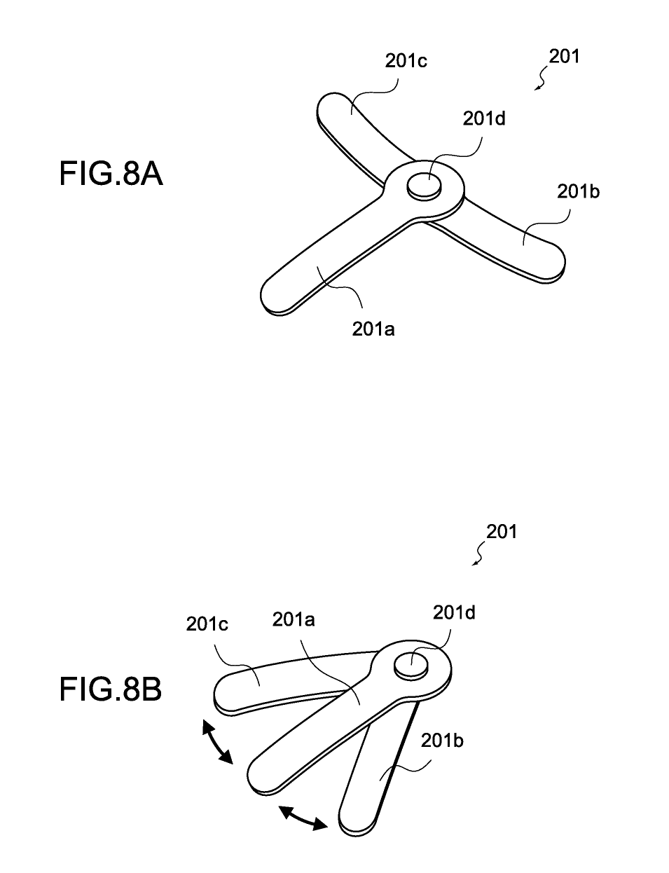

FIGS. 8A and 8B are schematic views showing a state in which the first headband section 201a, the second headband section 201b, and the third headband section 201c rotate. FIG. 8A shows the headband 201 in the portable shape. FIG. 8B shows the headband 201 in a state in which the headband sections 201a to 201c are rotated from the portable shape.

As shown in the figure, the headband 201 can be folded by rotating the first headband section 201a, the second headband section 201b, and the third headband section 201c around the rotating shaft 201d. With this, the portability can be further enhanced from the portable shape.

As described above, the headband 201 according to this embodiment is configured to be deformable between the use shape and the portable shape and further can be folded from the portable shape. With the headband 201 according to this embodiment, the portability of the brain wave measurement apparatus can be further enhanced.

Further, as in the first embodiment, the brain wave measurement apparatus according to this embodiment can enhance the convenience with the housing casing having a function of realizing the recovery condition.

The present disclosure is not limited to each of the above-mentioned embodiments. The present disclosure may be modified without departing from the gist of the present disclosure.

In each of the above embodiments, the brain wave measurement apparatus has been described as the biological signal measurement apparatus. However, the present disclosure is not limited thereto. The present disclosure is applicable to a measurement apparatus that is attached to the body of the user and measures biological signals of the user. The supporter is not limited to the headband and may be a supporter to be attached to the body of the user. Further, the vital sensors are also not limited to the brain wave acquiring electrodes and may be various sensors capable of acquiring the biological signals.

Further, in each of the above-mentioned embodiments, the recovery condition is applied to the headband. However, the recovery condition may be applied to the headband by a mechanism provided to the headband. Examples of such a mechanism may include an electrically heated wire incorporated in the headband and capable of heating the headband.

It should be noted that the present disclosure may also take the following configurations.

(1) A biological signal measurement apparatus, including:

a supporter made of a shape-memory material; and

a vital sensor configured to acquire a biological signal of a user, the vital sensor being attached to the supporter.

(2) The biological signal measurement apparatus according to Item (1), in which

the supporter memorizes a shape conforming to a shape of a body of the user.

(3) The biological signal measurement apparatus according to Item (1) or (2), in which

the supporter includes a headband to be mounted on a head of the user, and

the vital sensor includes a brain wave acquiring electrode configured to abut against the head of the user and acquire a brain wave of the user.

(4) The biological signal measurement apparatus according to any one of Items (1) to (3), in which

the headband includes
a first headband section extending from a forehead of the user to an upper portion of an occipital region of the user,
a second headband section being connected to the first headband section and extending from the upper portion of the occipital region of the user to a right mastoid region of the user orthogonally to the first headband section, and
a third headband section being connected to the first headband section and extending from the upper portion of the occipital region of the user to a left mastoid region of the user orthogonally to the first headband section.

(5) The biological signal measurement apparatus according to any one of Items (1) to (4), in which

the first headband section, the second headband section, and the third headband section are rotatably connected by a single rotating shaft.

(6) Biological signal measurement equipment, including

a supporter made of a shape-memory material, to which a vital sensor configured to acquire a biological signal of a user can be attached.

(7) A biological signal measurement apparatus set, including:

a biological signal measurement apparatus including
a supporter made of a shape-memory material, and
a vital sensor configured to acquire a biological signal of a user, the vital sensor being attached to the supporter; and

a housing casing configured to house the biological signal measurement apparatus and provide the supporter with a recovery condition of the shape-memory material.

The present disclosure contains subject matter related to that disclosed in Japanese Priority Patent Application JP 2012-125903 filed in the Japan Patent Office on Jun. 1, 2012 the entire content of which is hereby incorporated by reference.

It should be understood by those skilled in the art that various modifications, combinations, sub-combinations and alterations may occur depending on design requirements and other factors insofar as they are within the scope of the appended claims or the equivalents thereof.

What is claimed is:

1. A biological signal measurement apparatus, comprising:
a supporter made of a shape-memory material; and
a vital sensor configured to acquire a biological signal of a user, the vital sensor being attached to the supporter.

2. The biological signal measurement apparatus according to claim 1, wherein
the supporter memorizes a shape conforming to a shape of a body of the user.

3. The biological signal measurement apparatus according to claim 2, wherein
the supporter includes a headband to be mounted on a head of the user, and
the vital sensor includes a brain wave acquiring electrode configured to abut against the head of the user and acquire a brain wave of the user.

4. The biological signal measurement apparatus according to claim 3, wherein
the headband includes
a first headband section extending from a forehead of the user to an upper portion of an occipital region of the user,
a second headband section being connected to the first headband section and extending from the upper portion of the occipital region of the user to a right mastoid region of the user orthogonally to the first headband section, and
a third headband section being connected to the first headband section and extending from the upper portion of the occipital region of the user to a left mastoid region of the user orthogonally to the first headband section.

5. The biological signal measurement apparatus according to claim 4, wherein
the first headband section, the second headband section, and the third headband section are rotatably connected by a single rotating shaft.

6. Biological signal measurement equipment, comprising
a supporter made of a shape-memory material, to which a vital sensor configured to acquire a biological signal of a user can be attached.

7. A biological signal measurement apparatus set, comprising:
a biological signal measurement apparatus including
a supporter made of a shape-memory material, and
a vital sensor configured to acquire a biological signal of a user, the vital sensor being attached to the supporter; and
a housing casing configured to house the biological signal measurement apparatus and provide the supporter with a recovery condition of the shape-memory material.
